(19) United States
(12) Patent Application Publication
(10) Pub. No.: US 2026/0033837 A1
(43) Pub. Date: Feb. 5, 2026

(54) INTEGRATED PCM DRIVER

(71) Applicant: MURATA MANUFACTURING CO., LTD., Nagaokakyo-shi, Kyoto (JP)

(72) Inventors: Jeffrey A. DYKSTRA, WOODSTOCK, IL (US); Jaroslaw ADAMSKI, STREAMWOOD, IL (US); Edward Nicholas COMFOLTEY, SAN DIEGO, CA (US)

(21) Appl. No.: 18/997,044

(22) PCT Filed: Jul. 12, 2023
(86) PCT No.: PCT/US2023/027477

§ 371 Date: Jan. 18, 2025

Related U.S. Application Data
(63) Continuation of application No. 17/815,193, filed on Jul. 26, 2022, now Pat. No. 11,942,929.

(51) Int. Cl. A61B 17/12; A61B 17/00

(52) U.S. Cl. CPC A61B 17/12109; A61B 17/12022; A61B 17/12113; A61B 17/1214; A61B 17/12145; A61B 2017/0053; A61B 2017/1205; A61B 2017/12054; A61B 2017/12095; Y10T 29/49826

(57) ABSTRACT

Methods and devices to control PCM switches are disclosed. The described devices include PCM switch drivers and logic and control circuits, all integrated with the PCM and the associated heater on the same chip. Various architectures for the driver are also presented, including architectures implement feedback mechanism to mitigate variations from process, temperature, and supply voltage.

### CROSS-REFERENCE RELATED APPLICATIONS

The present application claims priority to U.S. patent application Ser. No. 17/815,193 filed on Jul. 26, 2022, the contents of which are incorporated herein by reference in its entirety.

### TECHNICAL FIELD

The present disclosure is related to phase change material (PCM) switches, more in particular to methods and devices used to integrate the PCM switches and their drivers onto the same chip.

### BACKGROUND

Phase change materials can generally be used to fabricate integrated circuit (IC) switches as they can be thermally transitioned between amorphous and crystalline states, showing several orders of magnitude change in resistivity. FIG. 1A shows the block diagram of a prior art PCM switch (100A) consisting of a volume of phase-change material (PCM) (102) and an adjacent heater (101), such as a resistor. The heater (101) produces the thermal power needed for transitioning the phase change material from one of the amorphous or crystalline states to the other.

An alternative arrangement to transition the phase change material is to inject current directly though the material. However such arrangement has been shown to be problematic for RF applications.

Currently, in most applications, PCM switches are programmed and driven using lab equipment. This is a rather bulky solution often offering lower performance and higher cost than desired. A more compact and higher performing solution such as a monolithic commercial switch product employing PCM switches is highly needed. In such a product, the PCM, driver and the associated logic circuitry would be integrated on the same chip. The present invention addresses this need.

### SUMMARY

The disclosed methods and devices implement an integrated driver to program (i.e., to switch ON/OFF) a phase change material (PCM) switch. The integrated driver interfaces the input from an application circuit to the PCM switch. The described devices provide the required programming waveforms to the PCM switch to transition the switch between the ON and OFF states.

According to a first aspect of the present disclosure, a PCM driving arrangement for programming a state of a phase change material (PCM) switch is provided, comprising: a PCM switch comprising a heater and a volume of phase-change material coupled to the heater; a driver circuit connected to the heater and configured to provide, to the heater, a first electrical pulse profile or a second electrical pulse profile, depending on a desired state to which the PCM switch is to be programmed; and a logic and control circuit coupled to the driver and configured to program the PCM switch by providing: a first control pulse to the driver when the PCM switch is to be programmed in an ON state in correspondence to the first electrical pulse profile, and a second control pulse to the driver when the PCM switch is to be programmed in an OFF state in correspondence to the second electrical pulse profile, wherein the PCM switch, the driver and the logic and control circuit are integrated on the same chip.

According to a second aspect of the present disclosure, a method of programming a state of a phase change material (PCM) switch is disclosed, the PCM switch comprising a heater and a volume of a PCM, the method comprising: providing a driver circuit; integrating the driver and the PCM switch on the same chip; applying a first control pulse to the driver circuit when the PCM switch is to be programmed in an ON state; and applying a second control pulse to the driver when the PCM switch is to be programmed on an OFF state.

According to a third aspect of the present disclosure, a method of programming a state of a phase change material (PCM) switch stack is disclosed, the PCM switch stack comprising a plurality of PCM switches arranged in a stacked configuration, the method comprising driving the plurality of PCM switches in separate time intervals, one or more PCM switches at a time

Further aspects of the disclosure are provided in the description, drawings and claims of the present application.

### DESCRIPTION OF THE DRAWINGS

FIG. 1A shows the block diagram of a prior art PCM switch.

FIG. 1B shows power profiles for driving PCM switch heaters.

FIG. 2 shows an exemplary driving arrangement for a PCM switch according to an embodiment of the present disclosure.

FIG. 3A shows an exemplary PCM switch driver when the driver is in the OFF state according to an embodiment of the present disclosure.

FIG. 3B shows an exemplary PCM switch driver when the driver is in the ON state according to an embodiment of the present disclosure.

FIG. 4 shows an exemplary timing diagram according to an embodiment of the present disclosure.

FIG. 5 shows an exemplary state diagram according to an embodiment of the present disclosure.

FIG. 6A shows an exemplary timing diagram of staggered control pulses according to an embodiment of the present disclosure.

FIG. 6B shows and exemplary driving arrangement of a PCM switch stack according to an embodiment of the present disclosure.

FIG. 7 shows an exemplary PCM switch driver according to an embodiment of the present disclosure.

FIG. 8A shows an exemplary PCM switch driver according to an embodiment of the present disclosure.

FIG. 8B shows an exemplary PCM switch driver according to an embodiment of the present disclosure.

Like reference numbers and designations in the various drawings indicate like elements.

### DETAILED DESCRIPTION

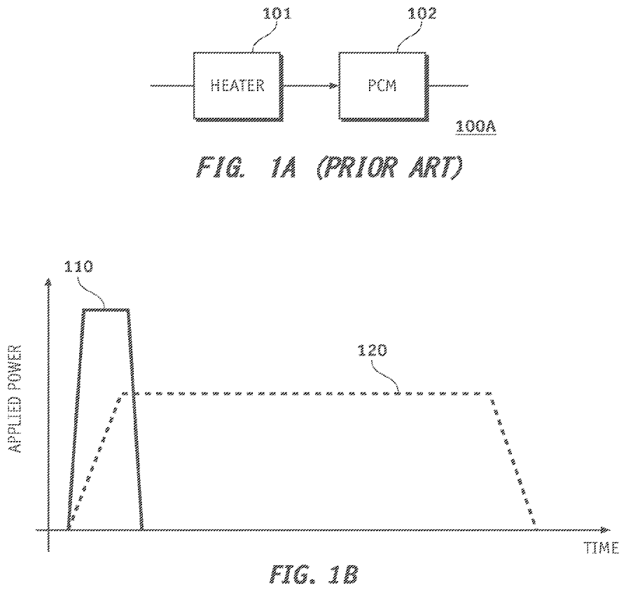

FIG. 1B shows two different electrical pulse profiles, in accordance with the present disclosure, for switching the resistivity states of the PCM (102) of FIG. 1A. Such electrical pulse profiles are applied to the heater (101) of FIG. 1A to generate different thermal profiles that result either in amorphizing the PCM (102) of FIG. 1A into a high resistance state (OFF or open) using a higher-power, short-period pulse (110), or crystalizing the PCM (102) into a low resistance state (ON or closed) using a lower-power, long-period pulse (120). Pulses (110, 120) may typically have pulse widths of 100 nsec and 1 usec respectively.

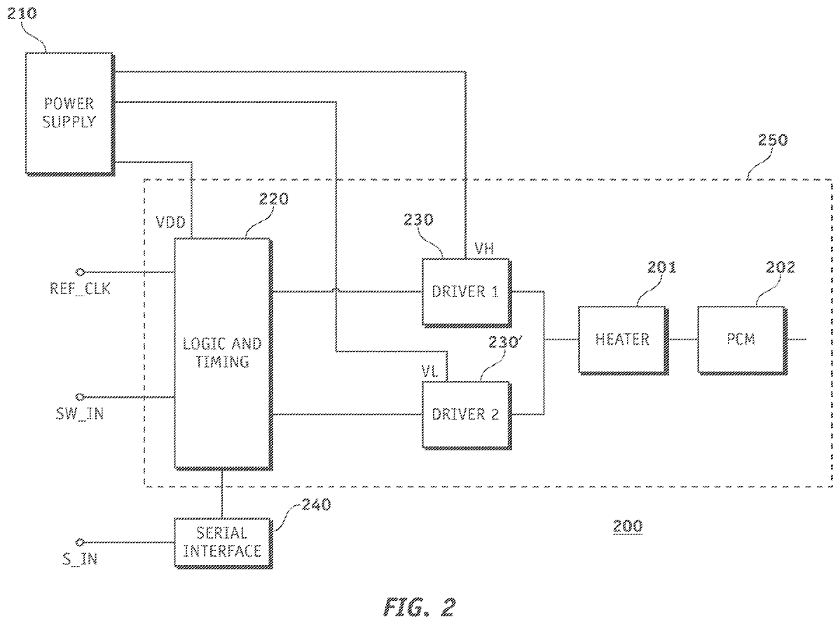

FIG. 2 shows an exemplary driving arrangement for a PCM switch (200) according to an embodiment of the present disclosure. The driving arrangement comprises a volume of PCM (202) a heater (201), a first driver (230), a second driver (230′), a logic and timing circuit (220), serial interface (240), and power supply module (210). For the purposes of the present disclosure, elements (201) and (202) will be collectively defined as PCM switch. As mentioned previously, different power profiles are implemented to drive a PCM switch. A pulse with a higher power and shorter pulse-width is implemented to put the PCM switch into the OFF state, while a different pulse with a lower power and longer pulse-width is implemented to turn the PCM switch into the ON state. Drivers (230, 230′) are used to drive heater (201) to turn PCM switch (201, 202) into the OFF/ON states respectively. As such, a higher bias voltage (VH) is provided to driver (230) by power supply module (210), while a lower bias voltage (VL) is provided to driver (230′) by power supply module (210). In an embodiment, a single driver, instead of two, may be implemented to generate the pulse waveforms for both the ON and OFF state of the PCM switch (201, 202). According to the teachings of the present disclosure, the widths of the pulses at the output of drives (230, 230′) to drive heater (201) can be programmable. In an embodiment all the constituents of the driving arrangement (200) are integrated on the same chip, as shown by the exemplary dotted box (250).

With further reference to FIG. 2, the logic and timing circuit (220) provides logic level pulses in correspondence with the state of the PCM switch (201, 202). Logic and timing circuit (220) receives digital reference clock input (REF_CLK) and digital control input (SW_IN), which are used to control the ON or OFF state of PCM switch (201, 202). In other words, based on the state of control input (SW_IN) and using digital reference clock input (REF_CLK), power profiles with proper amplitudes and widths in correspondence with the desired state of the PCM (202) are generated and fed to heater (201). As a result, PCM switch (021, 202) is transitioned between the ON and OFF states based on digital control input (SW_IN).

With continued reference to FIG. 2, power supply module (210) provides also the bias voltage (VDD) for logic and timing circuit (220). Switching arrangement (200) further comprises serial interface (240) connected to logic and timing circuit (220). Serial interface (240) includes an input (S_IN) and may be implemented using a serial peripheral interface (SPI) standard, a mobile industry processor interface (MIPI) standard or alike. The input control signal may also come from the serial interface.

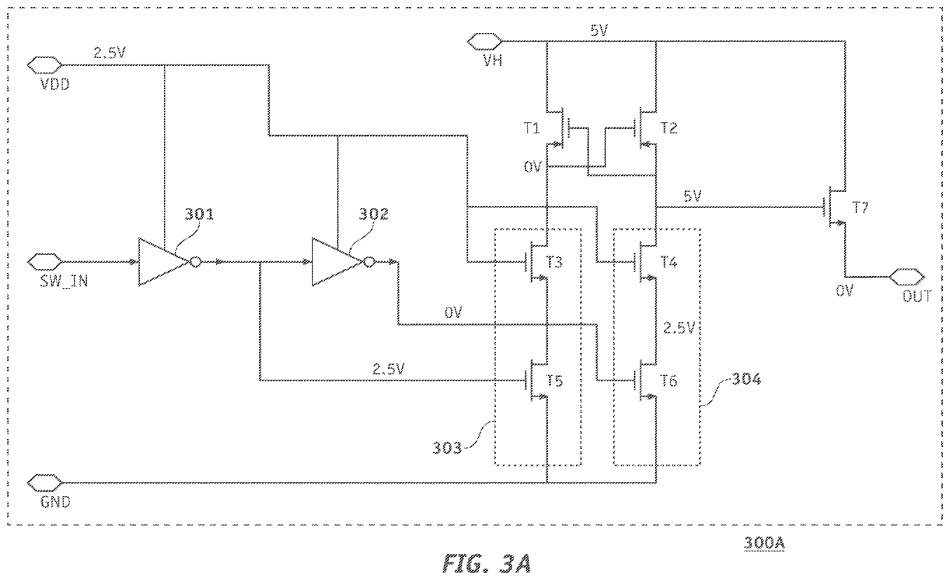

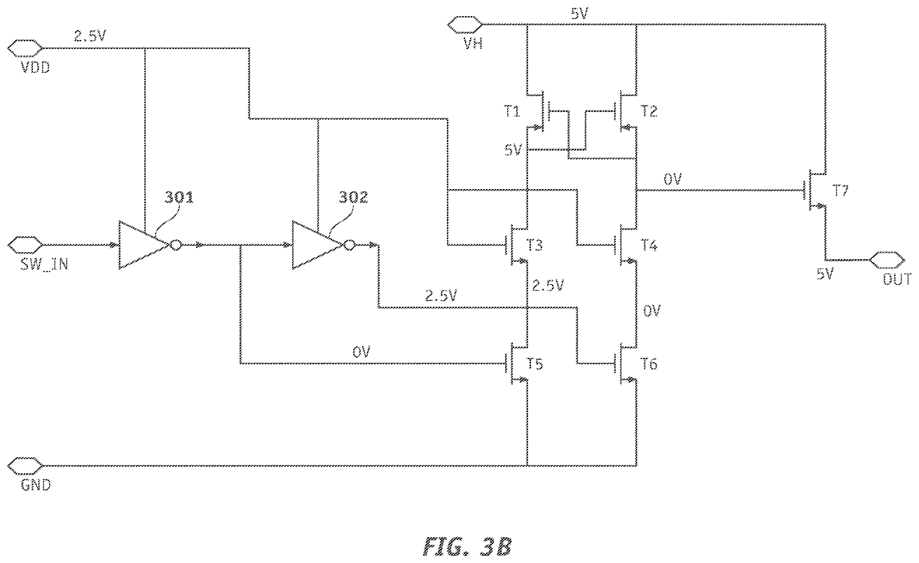

FIG. 3A shows an exemplary PCM switch driver (300A) representing an implementation of any of drivers (230, 230′) of FIG. 2 according to an embodiment of the present disclosure. Driver (300A) comprises a first driver switch stack (303) including transistors (T3, T5), and a second driver switch stack (304) including transistors (T4, T6). Driver switch stacks (303, 304) are each arranged in series with corresponding load device transistors (T1, T2), respectively. Also shown, is an output transistor (T7) delivering the voltage and current required by a downstream heater (e.g., heater (201) of FIG. 2) at output terminal (OUT). Driver (300A) further includes a serial arrangement of logic inverters (301, 302) receiving the control input (SW_IN). The logic inverters (301, 302) operate with bias voltage (VDD) and drive transistors (T5, T6) respectively Bias voltage (VH) is also provided to the driver similarly to what was described with regards to the embodiment of FIG. 2. According to the teachings of the present disclosure, the stacked arrangement shown in the figure allows for improved voltage handling. If desired, the transistor stacking can be increased or reduced according to transistor voltage handling capability and output drive required.

The voltage values shown in FIG. 3A represent the case where the PCM switch driver (300A) is in an OFF state. No current is being sent to the heater. Exemplary values for bias voltages (VDD, VH) may be 2.5V, 5V respectively. Voltage levels at various points of the driver (300A) are shown based on such exemplary bias values. On the other hand, FIG. 3B shows the same driver as in FIG. 3A, but this time with voltage values where the PCM switch driver is in the ON state. This is used to transition the PCM material to either the ON or OFF state depending on output amplitude and timing, as previously described.

With reference to FIGS. 3A-3B, transistors (T3, T4, T5, T6) may be NFETs and transistors (T1, T2, T7) may be PFETs. Based on the exemplary bias voltages of (2.5V, 5V) for bias voltages (VDD, VH), the NFETs and PFETs may be each designed to handle a voltage of at least (2.5V, 5V) respectively.

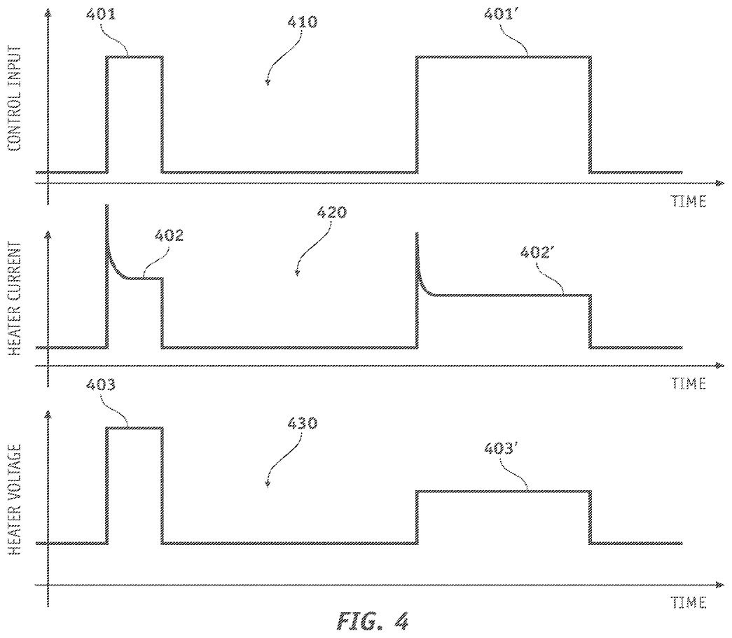

FIG. 4 represents exemplary timing diagrams related to the embodiment of FIG. 2. Signals (410, 420, 430) represent control input, heater current, and heater voltage vs. time respectively. Pulses (401, 402, 403) corresponds to the case where the PCM switch is driven or programmed to an OFF state while pulses (401′, 402′, 403′) represent the case where such switch is driven or programmed to an ON state.

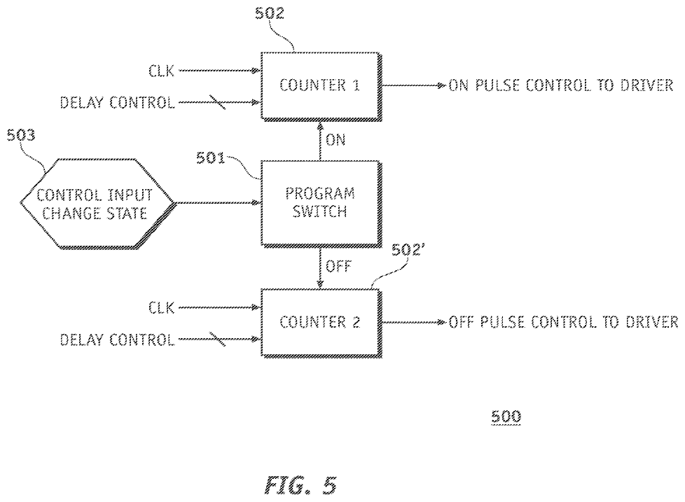

With reference to FIG. 2, operation of the logic and timing module (220) may be described as a state machine. FIG. 5 shows an exemplary state diagram (500) according to an embodiment of the present disclosure. Referring to both FIG. 2 and FIG. 5, the PCM switch (201, 202) of FIG. 2 may be programmed (501) to be in an ON or OFF state based on the state (503) of digital control input (SW_IN). When the PCM switch (201, 202) is transitioning to the ON state, first counter (502) will generate an ON pulse with a desired width to the driver (230) of FIG. 1, see also FIG. 3A. The desired width may be implemented by counting the number of periods in a reference clock (clk) which is an input to the first counter (502). Similarly, when the PCM switch (201, 202) is transitioning into the OFF state, second counter (502′) will generate an OFF pulse with a desired width to the driver (230′) of FIG. 1, see also FIG. 3B. Also in this case, the width of the OFF pulse may be achieved by counting the number of clock periods in the reference clock (clk) which is an input to the second counter (502′). The control inputs to the first and second counters may have different values.

As mentioned previously, pulses are needed to be applied to the heater to transition the PCM switch between one state and another. More in particular, higher power profiles need to be implemented when transitioning to the OFF state. This may be problematic when designing PCM switches arranged in a stack configuration for an improved voltage handling. In such stacks, each PCM requires its own separate heater to be programmed and changing the state of all the stacked PCMs at the same time may be taxing on the power supply. This imposes power supply design challenges for the applications using PCM switch stacks.

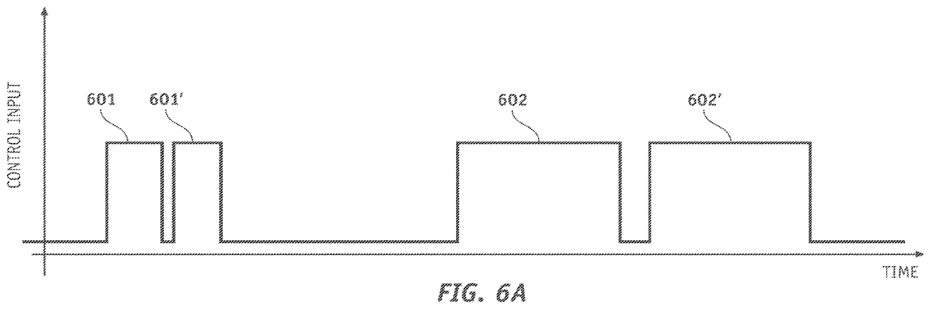

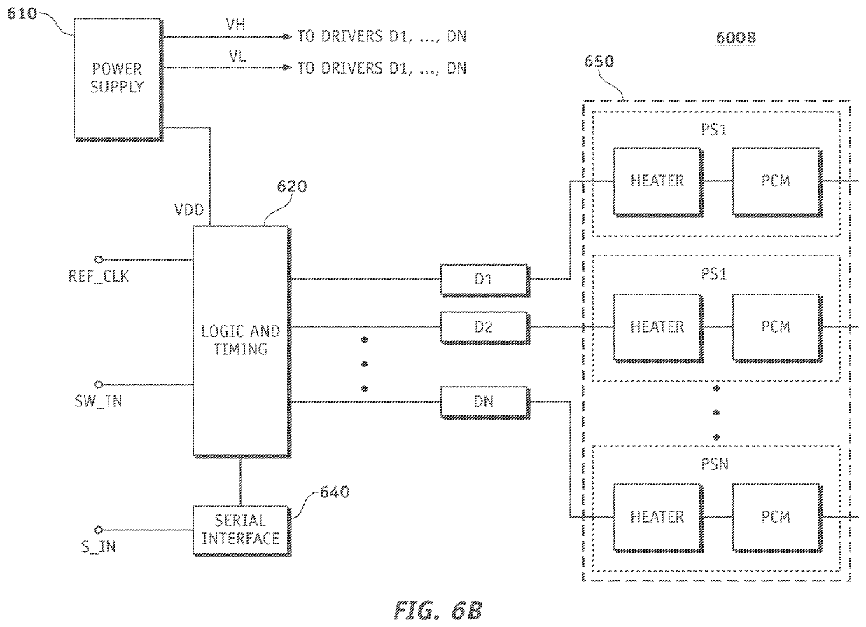

According to the teachings of the present disclosure, changing the state of the PCM switches within the stack may be performed in a staggered fashion, so that not all the PCM switches are changing state at the same time. As an example, this can be performed one PCM device at a time or several PCM devices at a time. As a result, the peak current drawn from the power supply can be reduced. In order to further clarify this teaching, reference is made to FIG. 6A showing the timing diagram presenting the control input pulses vs. time, when a staggered control of a PCM switch stack is adopted. Control pulses (601, 601′) are issued sequentially and they correspond to programming the OFF state of two of the PCM switches within the stack. Similarly, control pulses (602, 602′) are issued sequentially and they correspond to programming the ON state of two of the PCM devices within the stack. The timing diagram of FIG. 6A is an example illustrating the staggered control and it is understood that such concept can be extended to control sequentially all of the PCM switches within the stack, one or more at a time, until all the PCM switches within the stack have changed state in accordance with the control input.

The above teachings can be extended to an RF switch with a plurality of inputs and PCM devices and having at least two switch arms to transition OFF or ON. In this embodiment, the control signals may be staggered such that only one PCM device is being programmed at a given instant in time. If the switch arms include stacked PCM devices, each switch stack can be programmed at a given time interval, each PCM switch of the stack being programmed at a given instant.

By way of example, FIG. 6B shows an exemplary driving arrangement for a PCM switch stack (600B) representing an exemplary implementation of the above-disclosed teachings related to the embodiment of FIG. 6A. The driving arrangement comprises PCM switch stack (650), drivers (D1, . . . , Dn), control and logic circuit (620), power supply (610), and serial interface (640). PCM switch stack (600B) includes a plurality of PCM switches (PS1, . . . , PSn) arranged in a stacked configuration, wherein in each PCM switch comprises a heater and volume of PCM. Logic and timing circuit (620) provides control input to each of drivers (D1, . . . , Dn) in accordance with the teachings disclosed with regards to the embodiment of FIG. 6A, i.e., in a staggered fashion. As a result, each of the drivers (D1, . . . , Dn) provides driving pulses to a corresponding PCM switch (PS1, . . . , PSn), in correspondence with the state of each power switch (PS1, . . . , PSn). Power supply (610) provides high and low bias voltages (VH, VL) to each driver (D1, . . . , Dn). Bias voltages (VH, VL) correspond to the ON/OFF state of the PCM switches within the PCM switch stack (6500), respectively. With reference to both FIGS. 6A-6B, the states of PCM switches (PS1, . . . , PSn) may be changed, one or more PCM switch at a time, and in separate/staggered time intervals. This means that less instantaneous current is drawn from the power supply to transition switching stack (650) from one state to another, resulting in a simpler power supply design.

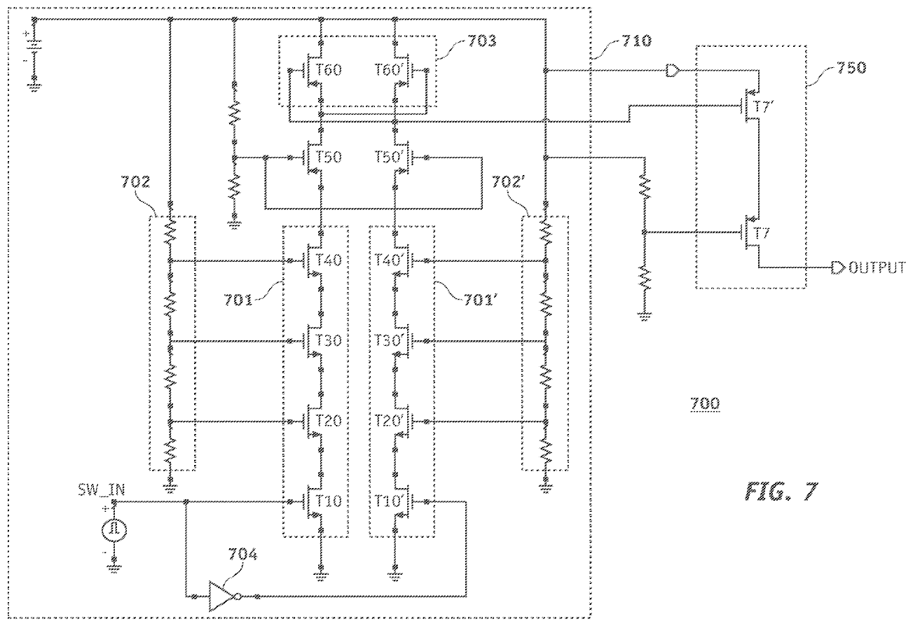

FIG. 7 shows an exemplary PCM switch driver (700) including a driver first stage (710) and a driver second stage (750) wherein the driver first stage (710) represents an implementation of any of drivers (230, 230′) of FIG. 2 according to an embodiment of the present disclosure. Terminal (OUTPUT) provides the input signal to the PCM (not shown). The embodiment of FIG. 7 is similar to the one shown in FIG. 3A, where the teachings of the disclosure are extended in order to handle larger supply voltages and output voltages. The embodiment of FIG. 7 could be beneficial in case of larger heater resistance values or in case a same driver drives a stack of heaters. Driver first stage (710) comprises a first driver switch stack (701) including transistors (T1, . . . , T4), and a second driver switch stack (701′) including transistors (T1′, . . . , T4′). Driver switch stacks (701,701′) are each arranged in series with corresponding transistors (T5, T5′), respectively. Driver first stage (710) further comprises quasi-latch (703) including transistors (T6, T6′). In particular, transistors T6 and T6′ are arranged to have positive feedback such that the resulting output signals transition quickly from one state to the other (i.e., driver OFF to ON), and the output voltage swing has the required amplitude to drive the next stage.

Also shown are resistive ladders (702, 702′) providing biasing to the gate terminal of the transistors within driver switch stacks (701, 701′), respectively. Control input (SW_IN) is directly applied to transistor (T1) while the inverted version of the control input (SW_IN) is applied to transistor (T1′) via inverter (704).

With further reference to FIG. 7, according to an embodiment of the present disclosure, transistors (T1, . . . , T4, T1′, . . . , T4′) may be NFETs and transistors (T5, T5′, T6, T6′) may be PFETs. As an example, such embodiment may be implemented using a bias voltage (VH) of 10V, in which case NFETs would have a breakdown voltage handling of 2.5V or better, and PFETs would have a breakdown voltage handling of 5V or better. The output of the circuit is at the drains of transistors (T5, T5′), in order to drive a stack of transistors similar to transistor (T7) shown in FIG. 3A.

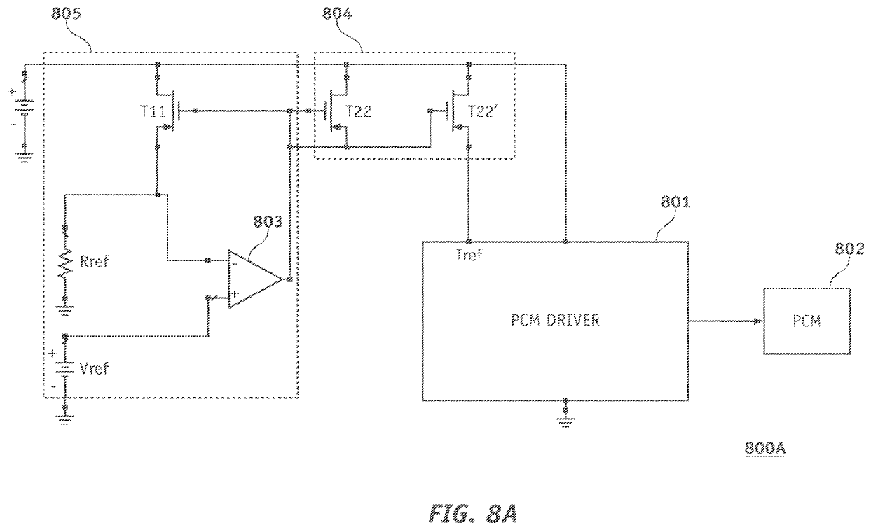

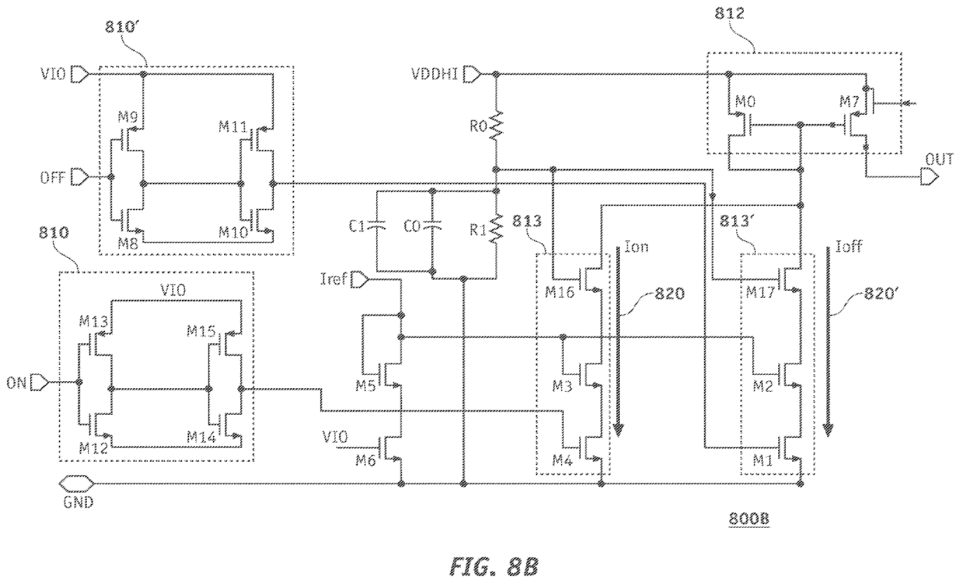

FIG. 8A shows an exemplary PCM switching arrangement (800A) according to an embodiment of the present disclosure, where a feedback mechanism is provided upstream of the PCM driver. In particular, PCM switching arrangement (800A) comprises PCM driver (801), PCM (802), feedback circuit (805), and transistors (T2, T2′) arranged as a current mirror (804). Feedback circuit (805) comprises operational amplifier (OP-amp) (803), reference resistor (Rref), reference voltage (Vref) and transistor (T1). Rref is intended to be identical to the heater resistor to be controlled. Such feedback circuit generates a desired current (Iref) through reference resistor (Rref) due to the voltages at both input terminals of the OP-amp (803) remaining the same at the desired reference voltage (Vref). Current mirror (804) essentially mirrors current (Iref) and feeds such current to PCM driver (801) to ensure the desired voltage and current are generated in the heater in correspondence with the desired state in which PCM (802) should be programmed. By virtue of implementing such feedback mechanism, variations from process, temperature, and supply voltage can be mitigated.

Implementation of the feedback mechanism of FIG. 8A will imply some design changes to the driver (801) with respect to the architectures shown in FIGS. 3A, 3B and 7. FIG. 8B shows an exemplary PCM driver (800B) according to an embodiment of the present disclosure. PCM driver (800B) represents an exemplary implementation of PCM driver (801) of FIG. 8A and includes switch stacks (813, 813′) that are driven by circuits (810, 810′) respectively, each provided to program the PCM switch (802) of FIG. 8A. When the PCM switch (802) is to be programmed in the ON state, the output of circuit (810) is HIGH and current Ion flows in switch stack (813), as indicated by arrow (820), while in the case when the PCM switch (802) is to be programmed in the OFF state, the output of circuit (810′) is HIGH and current Ioff flows in switch stack (813′), as indicated by arrow (820′).

FIG. 8B also shows current Iref as an additional input, in accordance with the teachings of FIG. 8A. Such current is fed to switch stacks (813) and (813′) through current mirror (M5, M3) to generate current Ion and current mirror (M5, M2) to generate current Ioff.

Reference current (Iref) is fixed by the feedback circuit. The values of currents Ion and Ioff are generated by the current mirror ratio generated by the ratio of M3 and M2 to M5.

PCM driver (800B) further comprises current mirror (812) that is used to generate the current required by the heater through terminal (OUT), and in correspondence with the ON and OFF states of the PCM switch.

The term “MOSFET”, as used in this disclosure, includes any field effect transistor (FET) having an insulated gate whose voltage determines the conductivity of the transistor, and encompasses insulated gates having a metal or metal-like, insulator, and/or semiconductor structure. The terms “metal” or “metal-like” include at least one electrically conductive material (such as aluminum, copper, or other metal, or highly doped polysilicon, graphene, or other electrical conductor), “insulator” includes at least one insulating material (such as silicon oxide or other dielectric material), and “semiconductor” includes at least one semiconductor material.

As used in this disclosure, the term “radio frequency” (RF) refers to a rate of oscillation in the range of about 3 kHz to about 300 GHz. This term also includes the frequencies used in wireless communication systems. An RF frequency may be the frequency of an electromagnetic wave or of an alternating voltage or current in a circuit.

With respect to the figures referenced in this disclosure, the dimensions for the various elements are not to scale; some dimensions have been greatly exaggerated vertically and/or horizontally for clarity or emphasis. In addition, references to orientations and directions (e.g., “top”, “bottom”, “above”, “below”, “lateral”, “vertical”, “horizontal”, etc.) are relative to the example drawings, and not necessarily absolute orientations or directions.

Various embodiments of the invention can be implemented to meet a wide variety of specifications. Unless otherwise noted above, selection of suitable component values is a matter of design choice. Various embodiments of the invention may be implemented in any suitable integrated circuit (IC) technology (including but not limited to MOSFET structures), or in hybrid or discrete circuit forms. Integrated circuit embodiments may be fabricated using any suitable substrates and processes, including but not limited to standard bulk silicon, high-resistivity bulk CMOS, silicon-on-insulator (SOI), and silicon-on-sapphire (SOS). Unless otherwise noted above, embodiments of the invention may be implemented in other transistor technologies such as bipolar, BiCMOS, LDMOS, BCD, GaAs HBT, GaN HEMT, GaAs pHEMT, and MESFET technologies. However, embodiments of the invention are particularly useful when fabricated using an SOI or SOS based process, or when fabricated with processes having similar characteristics. Fabrication in CMOS using SOI or SOS processes enables circuits with low power consumption, the ability to withstand high power signals during operation due to FET stacking, good linearity, and high frequency operation (i.e., radio frequencies up to and exceeding 300 GHz). Monolithic IC implementation is particularly useful since parasitic capacitances generally can be kept low (or at a minimum, kept uniform across all units, permitting them to be compensated) by careful design.

Voltage levels may be adjusted, and/or voltage and/or logic signal polarities reversed, depending on a particular specification and/or implementing technology (e.g., NMOS, PMOS, or CMOS, and enhancement mode or depletion mode transistor devices). Component voltage, current, and power handling capabilities may be adapted as needed, for example, by adjusting device sizes, serially “stacking” components (particularly FETs) to withstand greater voltages, and/or using multiple components in parallel to handle greater currents. Additional circuit components may be added to enhance the capabilities of the disclosed circuits and/or to provide additional functionality without significantly altering the functionality of the disclosed circuits.

Circuits and devices in accordance with the present invention may be used alone or in combination with other components, circuits, and devices. Embodiments of the present invention may be fabricated as integrated circuits (ICs), which may be encased in IC packages and/or in modules for ease of handling, manufacture, and/or improved performance. In particular, IC embodiments of this invention are often used in modules in which one or more of such ICs are combined with other circuit blocks (e.g., filters, amplifiers, passive components, and possibly additional ICs) into one package. The ICs and/or modules are then typically combined with other components, often on a printed circuit board, to form part of an end product such as a cellular telephone, laptop computer, or electronic tablet, or to form a higher-level module which may be used in a wide variety of products, such as vehicles, test equipment, medical devices, etc. Through various configurations of modules and assemblies, such ICs typically enable a mode of communication, often wireless communication.

A number of embodiments of the invention have been described. It is to be understood that various modifications may be made without departing from the spirit and scope of the invention. For example, some of the steps described above may be order independent, and thus can be performed in an order different from that described. Further, some of the steps described above may be optional. Various activities described with respect to the methods identified above can be executed in repetitive, serial, and/or parallel fashion.

It is to be understood that the foregoing description is intended to illustrate and not to limit the scope of the invention, which is defined by the scope of the following claims, and that other embodiments are within the scope of the claims. In particular, the scope of the invention includes any and all feasible combinations of one or more of the processes, machines, manufactures, or compositions of matter set forth in the claims below. (Note that the parenthetical labels for claim elements are for ease of referring to such elements, and do not in themselves indicate a particular required ordering or enumeration of elements; further, such labels may be reused in dependent claims as references to additional dements without being regarded as starting a conflicting labeling sequence).

1.-19. (canceled)

20. A method of programming a state of a phase change material (PCM) switch stack, the PCM switch stack comprising a plurality of PCM switches arranged in a stacked configuration, each PCM switch comprising a heater, the method comprising driving the plurality of PCM switches in separate time intervals, one or more PCM switches at a time.

21. The method of claim 20, further comprising transitioning each of the plurality of PCM switches between an ON state and an OFF state by applying a respective electrical pulse profile to the heater of that PCM switch during the corresponding time interval.

22. The method of claim 21, wherein the electrical pulse profile for transitioning to the OFF state comprises a first electrical pulse having a higher power and shorter pulse width, and the electrical pulse profile for transitioning the switch to the ON state comprises a second electrical pulse having a lower power and longer pulse width.

23. The method of claim 22, further comprising providing a reference clock input to a logic and control circuit that generates the separate time intervals, and selecting the pulse widths of the first and second electrical pulses by counting respective clock cycles from the reference clock input using the logic and control circuit.

24. The method of claim 23, wherein the logic and control circuit is integrated on the same chip as the plurality of PCM switches and is configured to receive a digital control input indicating whether each PCM switch is to be placed in the ON state or the OFF state.

25. The method of claim 20, wherein driving the plurality of PCM switches in separate time intervals comprises generating staggered control pulses using a plurality of driver circuits, each driver circuit corresponding to one of the plurality of PCM switches, wherein each control pulse enables the corresponding driver circuit while disabling driver circuits for all other PCM switches.

26. The method of claim 25, wherein each driver circuit is configured to generate an electrical pulse for placing the corresponding PCM switch in the ON state or the OFF state, the method further comprising supplying a first bias voltage to the driver circuit for generating the pulse for the OFF state, and a second bias voltage, lower than the first, for generating the pulse for the ON state.

27. The method of claim 20, further comprising using a current mirror to establish a reference current for the electrical pulses applied to the heater of each PCM switch.

28. The method of claim 27, wherein establishing the reference current comprises applying a feedback circuit including an operational amplifier, a reference resistor matched to the heater, and at least one transistor arranged to mirror a set current to each driver circuit corresponding to a PCM switch.

29. The method of claim 25, wherein each PCM switch is integrated with its corresponding driver circuit and the logic and control circuit in a single monolithic integrated circuit chip.

30. The method of claim 29, wherein each driver circuit includes a first transistor stack configured to drive the heater for the OFF state and a second transistor stack configured to drive the heater for the ON state, each transistor stack being activated in a non-overlapping time interval within the separate time intervals.

31. A system for programming a state of a phase change material (PCM) switch stack, the system comprising:
a plurality of PCM switches arranged in a stacked configuration, each PCM switch comprising a heater and a volume of phase-change material coupled to the heater;
a plurality of driver circuits, each driver circuit coupled to one of the PCM switches; and
a logic and control circuit coupled to the driver circuits,
wherein the logic and control circuit is configured to activate the plurality of PCM switches in separate time intervals, one PCM switch at a time, by providing control pulses to the respective driver circuits to transition each PCM switch between an ON state and an OFF state.

32. The system of claim 31, wherein each PCM switch and corresponding driver circuit is integrated on the same chip.

33. The system of claim 31, wherein each driver circuit is configured to provide, responsive to the control pulses:
a first electrical pulse profile having a lower power and longer pulse width to transition a corresponding PCM switch into the ON state; and
a second electrical pulse profile having a higher power and shorter pulse width to transition the corresponding PCM switch into the OFF state.

34. The system of claim 33, wherein the logic and control circuit comprises at least one programmable counter and receives a reference clock, the at least one programmable counter being configured to determine the pulse width of each electrical pulse profile by counting cycles of a reference clock.

35. The system of claim 31, further comprising a serial interface configured to supply digital control signals to the logic and control circuit indicating whether each PCM switch is to be driven to the ON or OFF state.

36. The system of claim 31, wherein the control pulses include a control pulse for the ON state and a control pulse for the OFF state, and wherein each driver circuit comprises:
a first transistor stack configured to receive the control pulse for the ON state; and
a second transistor stack configured to receive the control pulse for the OFF state, each transistor stack being arranged in series with at least one load device transistor.

37. The system of claim 31, further comprising:
a feedback circuit including an operational amplifier and a reference resistor, arranged to generate a reference current; and
a current mirror coupled to the feedback circuit and the plurality of driver circuits, the current mirror configured to mirror the reference current into each driver circuit,
wherein the reference current is selected such that process, temperature, and supply voltage variations are mitigated.

38. The system of claim 37, wherein the current mirror in each driver circuit is arranged to generate two different currents based on the reference current: a first current corresponding to the ON state and a second current corresponding to the OFF state.

39. The system of claim 33, wherein each heater comprises a heater resistor, and wherein the first and second pulse profiles produce different thermal power through said heater resistor to effect the desired ON or OFF state of the phase change material.
